(19) United States
(12) Patent Application Publication
(10) Pub. No.: US 2014/0331737 A1
(43) Pub. Date: Nov. 13, 2014

(54) GAS APPLICATION DEVICE FOR GAS MEASURING APPARATUSES, METHOD FOR TESTING GAS MEASURING APPARATUSES, AND CALIBRATION MEASURING APPARATUS FOR TESTING AND CALIBRATING GAS MEASURING APPARATUSES

(71) Applicant: Dräger Safety AG & Co. KGaA, Lübeck (DE)

(72) Inventors: Ingo Kaneblei, Lubeck (DE); Stefan Barten, Lubeck (DE)

(73) Assignee: Drager Safety AG & Co. KGaA a corporation

(21) Appl. No.: 14/361,075

(22) PCT Filed: Nov. 28, 2012
(86) PCT No.: PCT/EP2012/073839

§ 371 Date: May 28, 2014

(30) Foreign Application Priority Data
Nov. 28, 2011 (DE) 10 2011 119 568.1
Apr. 23, 2012 (DE) 10 2012 008 274.6

(51) Int. Cl. G01N 33/00

(52) U.S. Cl. CPC G01N 33/0006; G01N 33/0073; G01N 33/0011
USPC 73/1.06; 73/23.2

(57) ABSTRACT

A gas application device (2) for gas measuring apparatuses (5) has a test chamber device (58) for providing a test gas. A predetermined positive pressure is applied in the line system that connects the test chamber device (58) to one or more test-gas containers (PG) such that a constant test-gas supply (flow) to the test chamber device (58) can be ensured. A method for testing gas measuring apparatuses (5), and a calibration measuring apparatus for testing and calibrating gas measuring apparatuses (5) are also provided.

### CROSS REFERENCE TO RELATED APPLICATIONS

This application is a United States National Phase Application of International Application PCT/EP2012/073839 filed Nov. 28, 2012 and claims the benefit of priority under 35 U.S.C. §119 of German Patent Applications DE 10 2011 119 568.1 filed Nov. 28, 2011 and DE 10 2012 008 274.6 filed Apr. 23, 2012, the entire contents of which are incorporated herein by reference.

### FIELD OF THE INVENTION

The present invention pertains to a gas application device for gas measuring apparatuses with a test chamber device for providing a test gas, to a method for testing gas measuring apparatuses as well as to a calibrating gas measuring apparatus for testing and calibrating gas measuring apparatuses.

### BACKGROUND OF THE INVENTION

Calibrating stations for gas measuring apparatuses, which require a test or calibration gas and a purging gas, inert gas or zero gas, for monitoring the functions and calibrating gas measuring apparatuses, are known from the general state of the art. For regular testing of gas measuring apparatuses, the latter are placed into so-called calibrating stations, in which a plurality of gas measuring apparatuses are tested in test modules.

### SUMMARY OF THE INVENTION

An object of the present invention is to provide a gas application device for gas measuring apparatuses with a test chamber device for providing a test gas, a method for testing gas measuring apparatuses as well as a calibrating gas measuring apparatus for testing and calibrating gas measuring apparatuses, with which device and method a reliable test and a reliable feed of test gas to at least one gas measuring apparatus to be tested is possible.

According to the present invention, a gas application device for gas measuring apparatuses with a test chamber device for providing a test gas is provided, which has such a line system that a predetermined volume flow is set in the test chamber device, so that accurate setting of measuring apparatuses to be tested can be guaranteed.

One aspect of the present invention relates to providing a gas application device, with which an overpressure can be generated in the line system of the gas application device, which line system is intended for providing at least one test gas. A constant and continuous feed of a test gas with a predetermined volume flow (constant flow) to and into a test chamber device can be made possible for providing the test gas for a sensor device of a gas measuring apparatus.

According to one aspect of the present invention, a gas application device with a test gas connection device for connecting a test gas container containing a test gas is provided, wherein the gas application device has, for testing gas measuring apparatuses:
at least one test chamber device with a test chamber and with a gas measuring apparatus accommodating device for detachably coupling a gas measuring apparatus for detecting a property of a test gas flowing through the testing device by means of a test gas sensor device of the gas measuring apparatus;
a main line, which is connected with the test gas connection device;
a feed line, which fluidically connects the main line and a feed connection device of the test chamber device; and
a volume flow setting device installed in the feed line and especially a pressure throttling device for setting a gas provided from the main line of the test chamber, so that when a test gas flowing from the test gas container with a predetermined pressure, which is constant within preset limits, is provided, [a] predetermined test chamber volume flow that is constant within preset limits flows in the test chamber device.

The gas application device according to the present invention, the gas application system according to the present invention and the method according to the present invention for operating the gas application device according to the present invention and the gas application system according to the present invention has the following advantages:

In the prior-art calibration stations, the test modules can poll the necessary quantity of test gas necessary for testing the particular gas measuring apparatus via a pump system (demand flow principle). This pump system is in direct contact with the test gas and requires a pump with a regulating means to bring the demand flow valve into a position in which flow is possible. The demand flow valve can release the flow only when a low vacuum is present on the suction side of the demand flow valve. This vacuum is to be generated by an electronically regulated pump in order to obtain a constant flow. The individual test gas containers are usually provided with a demand flow valve. The demand flow valves on the test gas containers and an electronically regulated pump can be advantageously eliminated for generating a constant flow in the gas application device according to the present invention and in the gas application system according to the present invention.

Due to the fact that the test gas is provided with a predetermined overpressure in one of the main lines, the time needed for testing and calibration by means of a calibrating apparatus for testing gas measuring apparatuses can be reduced, because the volume flow is practically present immediately at the test chamber of the test chamber device and does not need to be drawn in first by means of a pump. Further, pumps can be eliminated for drawing in the test gas. In addition, a constant flow can be provided with the gas application device according to the present invention due to the overpressure of the test gas, as a result of which the ripple effect and a volume pulsation associated therewith of a measuring apparatus to be tested during the testing operation can be prevented from occurring. Since no pumps are used to draw the test gas to the test chambers of the test chamber devices, no additional pneumatic damping members are necessary. Since no pumps are necessary, the operation of the gas application device is associated with especially low noise. Due to the fact that a smaller number of components may be necessary for providing the test gas, weight and manufacturing costs are reduced. Further, the overpressure in the gas application device ensures that leaks in the line system cannot distort the measurement result, because no ambient air is drawn in, unlike in the case of calibrating gas measuring apparatuses in which the test gas is drawn in via pumps.

Provisions are made according to one embodiment of the gas application device according to the present invention for the gas application device to have a pressure-reducing device installed in the main line, so that the test gas is provided with predetermined pressure that is constant within preset limits between this gas application device and the volume flow setting device.

Provisions are made according to one embodiment of the gas application device according to the present invention for the gas application device to have a feed line switching device installed in the feed line for blocking or letting through test gas flowing from the test gas connection device to the feed line switching device.

Provisions are made according to one embodiment of the gas application device according to the present invention for the gas application device to have a feed line switching device installed in the main line for blocking or letting through test gas flowing from the test gas connection device to the feed line switching device.

Provisions are made according to one embodiment of the gas application device according to the present invention for installing an outlet line, in which a nonreturn valve is installed, on the test chamber of the test chamber device.

Provisions are made according to one embodiment of the gas application device according to the present invention for connecting an outlet line to the test chamber of the test chamber device and for connecting a differential pressure sensor to the main line and the outlet line.

Provisions are made according to one embodiment of the gas application device according to the present invention for connecting an outlet line to the test chamber of the test chamber device and for connecting a return line connecting the main line and the outlet line to the main line and to the outlet line, and for installing a feed line switching device in the return line.

According to another aspect of the present invention, a gas application device with at least two test gas connection devices is provided for connecting a test gas container each containing a test gas, which said test gas container has especially a pressure-reducing device connected thereto and a connection device each, wherein the gas application device has, for testing gas measuring apparatuses:

at least one test chamber device with a test chamber each;
an inlet line connected to a test gas connection device and connection-side switching devices for setting switching states with which blocking or letting through of test gas flowing from the respective test gas connection device can be set, wherein a connection-side switching device is installed fluidically in a respective inlet line;
a main line, which is connected to each of the inlet lines, so that the feed of test gas from one or more of the inlet lines into the main line can be set by means of the connection-side switching devices;
at least one feed line, one each of which connects the main line with a feed connection device of a test chamber device each; and
one volume flow setting device each fluidically installed in a plurality of feed lines or in each of the feed lines and especially a pressure-throttling device for setting a gas provided by the main line of the respective test chamber to a predetermined test chamber volume flow in the respective test chamber device or a pressure regulating valve, so that a predetermined test chamber device, into which a gas can be fed from a selectable test gas container of the test chamber device selected, can be selected.

Provisions are made according to one embodiment of the gas application device according to the present invention for connecting to each test chamber device an outlet line, into which a nonreturn valve is installed, and which is designed especially such that it allows the discharge of test gas from the respective test chamber only if a predetermined minimum test gas pressure has developed in the test chamber.

Provisions are made according to one embodiment of the gas application device according to the present invention for connecting to each test chamber device an outlet line each, to which an outlet main line each is connected, in which outlet main line a nonreturn valve is installed, which is designed especially such that it permits the discharge of test gas from the respective test chamber only when a predetermined minimum test gas pressure has developed in the test chamber.

Provisions are made according to one embodiment of the gas application device according to the present invention for fluidically installing in a plurality of feed lines or in each of the feed lines a commandable feed line switching device each for blocking or letting through the flow of test gas flowing from the main line to the respective test chamber device, so that a predetermined test chamber device, into which a gas can be fed from a selectable test gas container of the selected test chamber device, can be selected with a combination of a feed line switching device and a volume flow setting device, which combination is fluidically installed in one of the feed lines each.

Provisions are made according to one embodiment of the gas application device according to the present invention for the gas application device to have a second main line, which is connected to one of the test gas connection devices for introducing an inert gas and/or to a compressor device connected to a surrounding area containing ambient air for introducing ambient air into the second main line.

Provisions are made, according to one embodiment of the gas application device according to the present invention, for the gas application device to have a plurality of second feed lines connected to the second main line. The second feed lines are fluidically in connection with the test chamber of the test chamber device. A first feed line connected to the first main line is connected to a first inlet of a feed line switching device and the second feed line is connected to a respective second inlet of the feed line switching device. The outlet of the feed line switching device is connected via a test chamber feed line to the respective test chamber device. Blocking or letting through of test gas is provided as desired from the first main line or from the second main line, through the feed line switching device, can be performed as desired. A volume flow setting device is able to set volume flow in the first feed line (or in several first feed lines) for setting a gas being provided from the first main line of the test chamber of the respective test chamber device to a predetermined test chamber volume flow when test gas with a pressure that is constant within predetermined limits is provided in the first main line. The volume flow setting device is designed especially as a pressure-throttling device for setting a gas being provided from the main line of the respective test chamber to a predetermined test chamber volume flow in the respective test chamber device or as a pressure-regulating valve. A volume flow setting device is able to set volume flow in the second feed line (or in several second feed lines) for setting a gas being provided from the second main line of the test chamber of the test chamber device to a predetermined test chamber volume flow when test gas with a pressure that is constant within predetermined limits is provided in the second main line. The second volume flow setting device is designed especially as a pressure-throttling device for setting a gas being provided from the second main line of the respective test chamber to a predetermined test chamber volume flow in the respective test chamber device or as a pressure-regulating valve. With this, corresponding to the switching state of the feed line switching device, gas is provided from the first main line or from the second main line, as desired. The gas can be fed to the test chamber of the test chamber device with a test chamber volume flow, with a value of that flow depending on the pressure with which the gas is provided from the first main line or from the second main line for the feed line switching device.

Provisions are made according to one embodiment of the gas application device according to the present invention, for the feed line switching device to be designed as a 3-2-way on-off valve.

Provisions are made according to one embodiment of the gas application device according to the present invention for the gas application device to have a plurality of second feed lines, each of which is connected to the second main line and which are fluidically in connection with the test chamber of the test chamber device. The first feed line, connected to the first main line, is connected to an inlet of a first feed line switching device and one of the second feed lines is connected to an inlet of a second feed line switching device. The outlet of the switching device is connected via a test chamber feed line to a respective inlet of the respective test chamber of the test chamber device. Blocking or letting through of test gas, provided from the first main line or from the second main line, as desired, can take place, as desired, through the respective feed line switching device with the respective first feed line switching device and the respective second feed line switching device. A volume flow setting device sets volume flow in the first feed line (or in several first feed lines) for setting a gas being provided from the first main line of the respective test chamber device to a predetermined test chamber volume flow when test gas with a pressure that is constant within predetermined limits is provided in the first main line. The volume flow setting device is designed especially as a pressure-throttling device for setting a gas being provided from the main line of the respective test chamber to a predetermined test chamber volume flow in the respective test chamber device or as a pressure-regulating valve. A second volume flow setting device sets a volume flow of the second feed line (or in several second feed lines) for setting a gas being provided from the second main line of the respective test chamber device to a predetermined test chamber volume flow when test gas with a pressure that is constant within predetermined limits is provided in the second main line. The second volume flow setting device is designed especially as a pressure-throttling device for setting a gas being provided from the main line of the respective test chamber to a predetermined test chamber volume flow in the respective test chamber device or as a pressure-regulating valve. Corresponding to the switching state of the feed line switching device, gas provided from the first main line or from the second main line, as desired, can be fed to the test chamber of the test chamber device with a test chamber volume flow with a flow value that depends on the pressure with which the gas is provided from the first main one or from the second main line for the feed line switching device.

Provisions are made according to one embodiment of the gas application device according to the present invention for the gas application device to have pressure-reducing devices, at least one of which is installed fluidically in an input line connected to a respective test gas connection device.

Provisions are made according to one embodiment of the gas application device according to the present invention for the switching devices for setting switching states with which blocking or letting through of test gas flowing from the respective test gas connection device can set, and/or the pressure-reducing devices installed in the respective inlet line to be installed in a housing part or frame part of a master module, so that the switching devices are integrated in a master module that is uniform in terms of hardware.

According to a further aspect of the present invention, a gas application system with a gas application device according to an embodiment of the present invention is provided, wherein the gas application system has a control device which is functionally connected with at least a plurality of the connection-side switching devices, so that the gas container from which gas is fed to a respective main line is selectable, and/or which is functionally connected with at least a plurality of the feed line switching devices or the feed line switching devices connected to both of the main lines, so that gas can be fed from at least one gas container to one or more of the test chamber devices as desired.

According to a further aspect of the present invention, a method for testing gas measuring apparatuses is provided, having the following steps:
Providing a test gas at a test gas connection device for connecting a test gas container containing a test gas;
Reduction of the test gas pressure to a predetermined pressure by means of a pressure-reducing device, wherein especially the predetermined constant pressure is maintained with a range of variation of up to 10%;
Switching to flow at least one first feed line (45, 45.1 through 45.10), which is connected to a main line (201) and to a test chamber device (58), so that the test gas provided in the main line (201) can flow into the test chamber of the test chamber device (58);
Throttling the at least one feed line, which is connected to at least one main line and to a test chamber device, so that the test gas provided in the respective main line flows into the test chamber of the test chamber device with a predetermined volume flow.

Provisions are made according to one embodiment of the method according to the present invention for testing gas measuring apparatuses for the predetermined gas to be admitted to the test chamber of the test chamber device over a predetermined time and/or until a predetermined test chamber pressure has built up in the test chamber of the test chamber device.

Provisions are made according to one embodiment of the method according to the present invention for testing gas measuring apparatuses for an opening of at least one feed line, which is connected to a main line, to take place, so that test gas is provided in the main line with a predetermined pressure.

Provisions are made according to one embodiment of the method according to the present invention for testing gas measuring apparatuses for the test gas in the test chamber of the test chamber device to be removed when a predetermined gas admission time has elapsed and/or a predetermined gas admission parameter and especially a gas concentration is present in the test chamber and especially a minimum time is present.

According to a further aspect of the present invention, a calibrating gas measuring apparatus for testing and calibrating gas measuring apparatuses is provided, which has a gas application device according to one embodiment of the present invention.

Further advantageous embodiments of the present invention, as well as exemplary embodiments for them will be explained in more detail below in connection with the drawing figures enclosed. Functionally similar parts or components shown in different figures are designated in these figures partly with the same reference numbers. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a pneumatic circuit diagram of a first embodiment of a gas application device according to the present invention for connecting a test gas cylinder;

FIG. 1b is a pneumatic circuit diagram of a second embodiment of a gas application device according to the present invention for connecting a test gas cylinder;

FIG. 2 is a pneumatic circuit diagram of a third embodiment according to the present invention of the gas application device of a modular design with at least one operating/master module and a plurality of testing apparatus modules for receiving a gas measuring apparatus to be tested;

FIG. 3 is a pneumatic circuit diagram of a fourth embodiment according to the present invention of the gas application device of a modular design with at least one operating/master module and a plurality of testing apparatus modules for receiving gas measuring apparatuses to be tested;

FIG. 4 is a pneumatic circuit diagram of a fifth embodiment according to the present invention of the gas application device of a modular design with at least one operating/master module and a plurality of testing apparatus modules for receiving a gas measuring apparatus to be tested;

FIG. 5 is a pneumatic circuit diagram of a sixth embodiment according to the present invention of the gas application device of a modular design with at least one operating/master module and a plurality of testing apparatus modules for receiving a gas measuring apparatus to be tested;

FIG. 6 is a schematic view of a pressure profile curve in a pressure-time coordinate system, which indicates the admission pressure of a plurality of test gas containers, which was received with a differential pressure sensor in the gas application device; and

FIG. 7 is a schematic view of a plurality of pressure profile curves, in a pressure-time coordinate system, of the expected curve describing a pressure drop in the feed lines in the gas application device according to the present invention.

### DESCRIPTION OF THE PREFERRED EMBODIMENTS

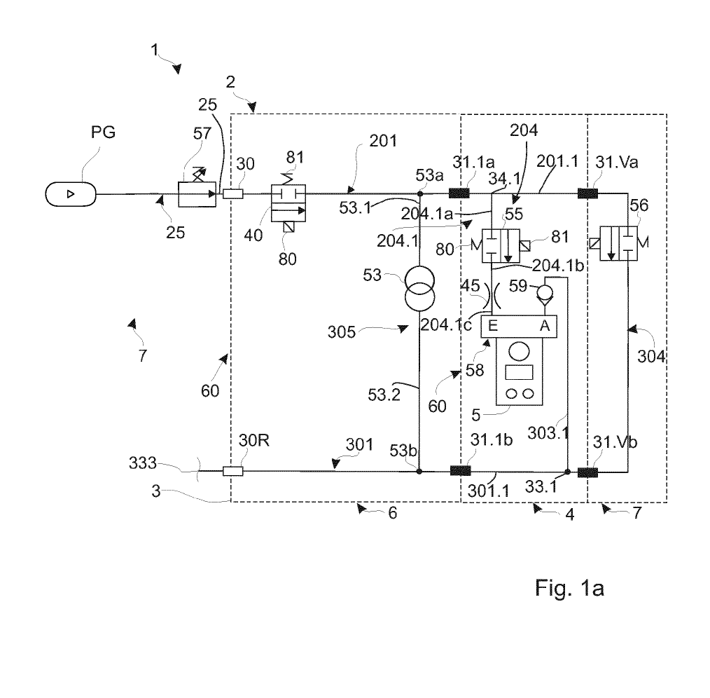

Referring to the drawings in particular, FIGS. 1a and 1b show each a pneumatic circuit diagram of an embodiment according to the present invention of a gas application device 2, which is intended for use in a device 1 for testing at least one gas measuring apparatus 5.

The gas application devices 2 shown in FIGS. 1a and 1b have each a test gas connection device 30 for connecting a test gas container PG containing a test gas. The test gas container PG may be especially a mobile test gas container PG. A connection device, not shown in FIG. 1a, with which an outlet device of the test gas container PG can be connected to or uncoupled from a connection line 25. The test gas container PG may also be a test gas container PG installed permanently in space, from which a connection line 25 installed permanently in space extends. The interior of the test gas container PG is connected in both embodiments with the interior of the respective connection line 25, so that gas contained in the test gas container PG can flow out of same via the connection line 25 when the test gas container PG is connected to the test gas connection device 30. The connection line 25 is connected with a main line 201 of the gas application device 2 via a test gas connection device 30.

According to one embodiment of the gas application device 2 according to the present invention as shown in FIG. 1a, a pressure-limiting valve 57 is installed in the connection line 25 downstream of the direction of flow of the gas from the test gas container 50. The function of the pressure-limiting valve 57 is to ensure that the pressure of gas that is flowing from the test gas container PG and is present in the line 25 behind the pressure-limiting valve 57 when viewed from said test gas container PG does not exceed a maximum pressure value and especially that a pressure of the gas that is equal to the maximum pressure value will become established in the line 25 behind the pressure-limiting valve 57 in case of a sufficient pressure of the gas in the test gas container 10 that exceeds this maximum pressure.

A main line 201 of the gas application device 2, in which a pressure with the maximum pressure value is present because of the pressure-limiting valve 57 in the connection line 25 when gas is flowing from the gas container 50 into the main line 201, is connected to the test gas connection line 30.

The pressure provided in a completely filled test gas container PG at the gas application device 2 according to the present invention may be, for example, in the range between 30 bar and 250 bar. The limitation to the maximum pressure value may be provided especially to a pressure value that may be between 0.2 bar and 5 bar and equals, e.g., 0.3 bar. The pressure of the gas flowing from the test gas container PG decreases with decreasing filling of the test gas container PG. The pressure-reducing valve 57 may be designed according to one embodiment of the present invention such that the pressure prevailing in the main line 201 can be set with this.

The connection line 25 of the gas container PG is thus connected to the main line 201 of the gas application device 2 by means of the test gas connection device 30.

The embodiment of the gas application device 2 shown in FIG. 1b is intended especially for eliminating the installation of a pressure-reducing valve (designated by reference number 57 in FIG. 1a) in the connection line 25, so that gas flowing from a test gas container PG flows through the test gas connection device 30 approximately with a pressure that is contained in the test gas container PG at an actual time. This means that the gas flows in the area of the connection device 30 in case of a correspondingly filled test gas container PG with a relatively high pressure into the main line 201. A pressure-limiting valve 52, with which the pressure of the gas is limited to a maximum pressure and optionally reduced, so that the gas flowing through the module connection device 31.1a and especially into the feed line 204.1 can have only a maximum pressure value, is integrated or installed for this purpose in the main line 201 between the test gas connection device 30 and the module connection device 31.1a.

Provisions may also be made according to the present invention for installing or integrating in the connection line 25 a pressure-limiting valve 57 for limiting and optionally reducing the pressure to a first pressure value, with which the gas flows from a test gas container PG connected to the gas application device 2 according to the present invention into the main line 201 or is provided from the test gas container PG to the main line 201, and for additionally installing or integrating, according to the embodiment according to FIG. 1b, a pressure-limiting valve 52 in the main line 201 between the test gas connection device 30 and the module connection device 31.1a for limiting and optionally reducing the pressure to a second pressure value, so that the gas is provided to the section 201.1 of the main line 201 and hence to the connection device 34.1 with a predetermined pressure after this two-stage pressure reduction.

Pressure-limiting valves 52 that are specifically suitable for the respective pressure reduction, which also permit, in particular, a more accurate setting of the pressure with which the test gas is provided to the section 201.1, can be used for the respective pressure reduction due to this two-stage pressure reduction.

The device limits of the embodiments of the gas application device 2 shown in FIGS. 1a and 1b are indicated by the largest rectangle, which is indicated by broken lines 60 and contains three partial rectangles, which are designated by the reference numbers 6, 4, 7. A rectangle designated by reference number 6 and formed with lines 60 may be part of an operating module or master module and a rectangle designated by the reference number 4 and formed with lines 60 may be part of a testing apparatus module. A rectangle designated by reference number 7 and formed with lines 60 may be a recirculating module 7, which is optionally provided according to the present invention.

The operating module or master module 6, which assumes essentially the control of the test modules in a preferred embodiment of the gas application device 2 or device for testing and calibrating gas measuring apparatuses, is formed in the embodiment shown in FIG. 1a or 1b from at least one first test gas connection device 30, to which the connection line 25 of the gas container 50 can be connected, and the connection-side switching device 40, especially in the form of a 2/2-way valve, which can be reset in the embodiment being shown into the starting position shown by means of a switch 80 connected functionally with the control device, especially in an electromagnetically actuatable manner, and by means of an adjusting spring 81. The connection-side switching device or the 2/2-way valve 40 is shown in a switching state in which the flow of gas through this valve and thus the flow of gas in the main line 201 are blocked. By means of a control device functionally connected with the switch 80, the switch 80 can be moved into a closed position, in which the switch 80 sets the switching device 40 into a state in which the switching device 40 blocks the flow of test gas through the line 201, and into an open position, in which the switch 80 sets the switching device 40 into a state in which the switching device 40 blocks a flow of test gas through the line 201. Provisions are made according to one embodiment of the present invention for the switch 80 to assume the closed position in a prestressed or preset state, so that if no command signal is transmitted from the control device to the switch 80, the latter is in the closed position and blocks the flow of test gas through the main line 201.

The embodiments of the gas application device 2 according to the present invention shown in FIGS. 1a and 1b have a differential pressure sensor 53, which is connected with the main line 201 via sensor lines 53.1 and 53.2, especially fluidically via a connection 53a, and with the recirculating line 301 via a connection 53b such that the differential pressure arising from the pressure present in the main line and the pressure present in the recirculating line 301 can be measured.

The embodiments of the gas application device 2 according to the present invention shown in FIGS. 1a and 1b have a test chamber device 58 with a test chamber (not shown in the figures) and with a gas measuring apparatus accommodating device (not shown in the figures) for detachably coupling a gas measuring apparatus 5 with same for detecting a property of a test gas flowing through the test chamber of the test chamber device by means of a test gas sensor device (not shown in the figures) of the gas measuring apparatus 5. The reference “E” always designates an inlet or an inlet device of the test chamber of the respective test chamber device and the reference “A” always designates an outlet or an outlet device of a test chamber of a respective test chamber device 58 in the representation of the respective test chamber device in the figures. A feed line, which branches off from the main line 201, opens into the inlet or an inlet device of the test chamber. The outlet or outlet device A may open directly into the area surrounding the gas application device 2 or into a receiving container (not shown) according to one embodiment of the present invention. An outlet line 303, through which gas flowing through the test chamber can flow out of said test chamber, is connected to the outlet A in the embodiment of the gas application device 2 shown in FIG. 1a.

The embodiments of the gas application device 2 according to the present invention shown in FIGS. 1a and 1b have a recirculating line 301, into which the outlet line 303 opens and which may especially be connected to a connection device 30R. Especially a connecting line or connection line 333, to which, for example, a collection container may be connected, may be connected to the respective connection device 30R. A test gas arriving from the test chamber and flowing through the connection line 333 can thus flow, for example, into the collection container in such an embodiment.

FIGS. 1a and 1b show only one test module 4 each, which is connected to a section of the main line 201 connected with the connection line 25 or to a master module or control module 6 by means of connections 31.1a and 31.1b, with which the main line and the recirculating line 301 is composed from line sections. The test module 4 with the test chamber device 58 can thus be connected to the operating/master module 6 by means of module connection devices 31.1a and 31.1b. The device limits of the test module 4 are likewise indicated by the broken line 60. A section 201.1 of the main line 201 and a section 301.1 of the recirculating main line 301 extend in the test module 4 according to the present invention. At least one test chamber feed line 204, which branches off from a branch or at a connection point 34.1 from the respective section 201.1 of the main line 201 and is connected to the test chamber inlet device E of the test chamber device 58 for receiving a test gas, is connected to the main line 201.

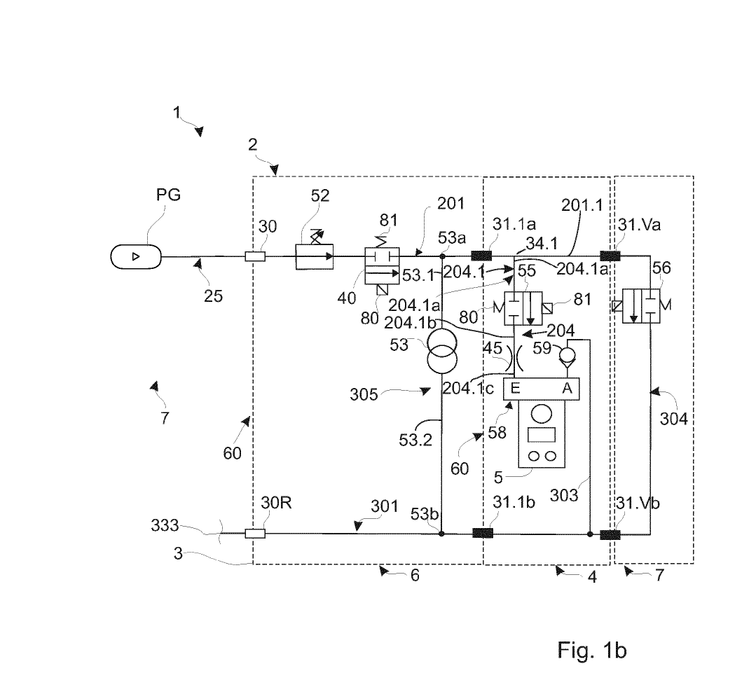

The embodiments of the gas application device 2 according to the present invention shown in FIGS. 2, 3, 4 and 5 have each a plurality of test chamber devices 58.1, 58.2, 58.10, to which a gas measuring apparatus 5 each can be connected, for which the respective test chamber device 58, 58.1, 58.2, 58.10 has a corresponding connection device, not shown in the figures, or gas measuring apparatus accommodating device for the mechanical and fluidic, especially detachable connection of a gas measuring apparatus 5.

According to one embodiment of the present invention, each test chamber device 58, 58.1, 58.2, 58.3, 58.10 may be modularly integrated as a component of a respective test module 4.1, 4.2, 4.3 or 4.10 in the gas application device 2 and connected to other modules, so that a plurality of test modules 4.1, 4.2, 4.3, 4.10 can be connected to one another in a chain-like manner. It can be stated in summary with the reference numbers shown as a whole in FIGS. 2 through 5 for the embodiments of the gas application device 2 according to the present invention shown in FIGS. 2 through 5 that the respective test module 4.1, 4.2, 4.3, 4.10 has:

a main line section 201.1, 201.2, 201.3, 201.10 each,

a feed line 204.1, 204.2, 204.3, 204.10 each, which is connected with a main line connection device 34.1, 34.2, 34.3, 34.10 to a respective section 201.1, 201.2, 201.3, 201.10 of a main line 201 and to the test chamber inlet device E of a respective test chamber device 58.1, 58.2, 58.3, 58.10 (analogously to the test chamber device 58 according to FIG. 1a or 1b) in order to provide test gas for the test chamber device 58.1, 58.2, 58.3, 58.10,

optionally an outlet line 303.1, 303.2, 303.3, 303.10, which is connected to the test chamber outlet device A of the respective test chamber device 58, 58.1, 58.2, 58.3, 58.10 and which is connected via a recirculating line connection device 32.1, 32.2, 32.3, 32.10 each to a respective section 301.1, 301.2, 301.3, 301.10 of the recirculating line 301, wherein provisions may be made for installing or integrating a nonreturn valve 59.1, 59.2, 59.3, 59.10 each in the outlet lines 303.1, 303.2, 303.3, 303.10 in order to prevent gas from being able to flow back into the test chamber, and

two connection devices 31.1a and 31.1b or 31.2a and 31.2b or 33.1a and 33.1b or 31.10a and 31.10b per test module 4.1, 4.2, 4.3, 4.10 (reference numbers corresponding to the respective figure), with which said connection devices a respective section 201.1, 201.2, 201.3, 201.10 of the main line 201, on the one hand, and a respective section 201.1, 201.2, 201.3, 201.10 of the main line 201 can be connected to a connection line 201a or to another section 201.1, 201.2, 201.3, 201.10 of the main line 201.

The test chamber device 58 or 58.1 or 58.2 or 58.3 or 58.10 is always designed such that it has a test chamber inlet device E, a test chamber (not shown) and a test chamber outlet device A such that test gas having entered the test chamber inlet device E can flow through the test chamber and through the test chamber outlet device A into an outlet line 303.1, 303.2, 303.3, 303.10. The outlet line 303.1, 303.2, 303.3, 303.10 is connected via a line connection 33.1 to the recirculating line 301 and especially a corresponding section 301.1, 301.2, 301.3, 301.10 of the recirculating line 301, so that the test gas can flow through the recirculating line 301 into a connection line 333 for its further use. A gas measuring apparatus 5 is connected to or mounted on the gas measuring apparatus accommodating device for detecting and testing a predetermined property of the sensor device of the gas measuring apparatus 5, so that a predetermined test gas provided by a respective test gas container flows through the gas application device 2 according to the present invention at a predefined flow rate or with a predetermined volume flow. For example, the sensor device of a testing apparatus connected to a test chamber device can be tested in this manner, because the test gas has known properties and, for example, an O₂ concentration or an H₂S concentration, and a desired value, with which a sensor value detected by the sensor device of the respective connected gas measuring apparatus 5.1, 5.2, 5.10 is compared as an actual value for the corresponding property to be tested as a test, is assigned as an already known property to these properties before a respective setting or testing process. A deviation or difference of the actual value from the desired value can be determined from this and an evaluation or improved setting of the respective testing apparatus 5.1, 5.2, 5.10 tested can be performed on the basis of the difference determined.

The embodiments of the gas application device 2 may have a connecting line or recirculating line 304 each, which may be connected to the main line 201, e.g., via a connecting device 31.Ra and to a recirculating line 301 provided in the gas application device 2, e.g., via a connecting device 31.Rb. A recirculation switching device 56, especially in the form of a 2/2-way valve, which can be switched, e.g., by means of a control device functionally connected with it, into a blocked state, in which the flow of gas through the recirculation switching device 56 is blocked, and into a flow state, in which a flow of gas through the recirculation switching device 56 is permitted, may be optionally provided in the connecting line or recirculating line 304. The recirculating line 301, the connection devices 31.Ra and 31.Rb and the recirculation switching device 56 may form a gas recirculation module designated by reference number 7.

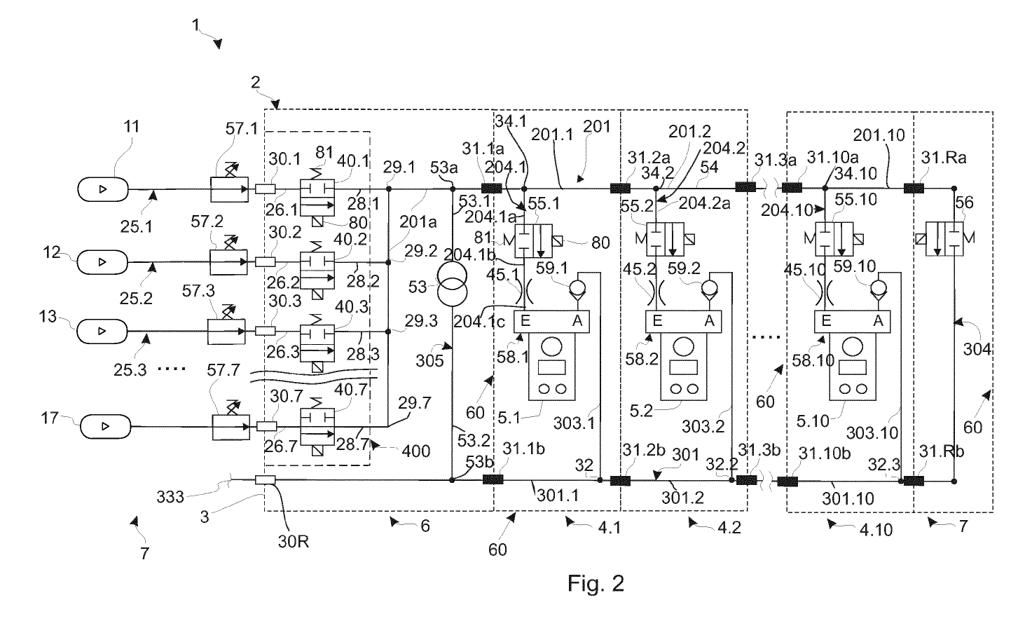

The following components are fluidically installed in each feed line 204.1, 204.2, 204.3, 204.10 in the embodiments of the gas application device 2 according to the present invention, which are shown in FIGS. 2 and 3:

A volume flow setting device or pressure-reducing device 45.1, 45.2, 45.3, 45.10 and especially in the function of a pressure-throttling device for setting a gas provided from the respective section 201.1, 201.2, 201.3, 201.10 of the main line 201 of the respective test chamber to a predetermined test chamber volume flow in the respective test chamber device 58.1, 58.2; 58.10 or especially a pressure regulating valve functionally connected with the control device for setting a gas provided from the respective section 201.1, 201.2, 201.3, 201.10 of the main line 201 of the respective test chamber to a test chamber volume flow present or commanded by the control device,

At least one feed switching device 55.1, 55.2, 55.3, 55.10, which can be commanded by a control device, e.g., in the form of a directional control valve in several of or all feed lines 204.1, 204.2, 204.3, 204.10, and, in the particular embodiment shown, a 2/2-way valve, which may be functionally connected with a control device for blocking or letting through the flow of test gas flowing from the main line 201 to the respective test chamber device 58.1, 58.2, 58.3, 58.10.

It is achieved with this arrangement that a predetermined test chamber device 58.1, 58.2, 58.3, 58.10, into which a gas can be fed from a selectable test gas container of the selected test chamber device 58.1, 58.2; 58.10, can be selected with a combination of a feed line switching device 55.1, 55.2, 55.3, 55.10 and a volume flow setting device 45.1, 45.2, 45.3, 45.10, which said combination is fluidically installed in one of the feed lines 204.1, 204.2, 204.3, 204.10, and setting of a gas provided from the main line 201 of the respective test chamber to a predetermined test chamber volume flow in the respective test chamber device 58.1, 58.2, 58.3, 58.10 is guaranteed.

Provisions may be made, in particular,

for the feed line switching devices 55.1, 55.2, 55.3, 55.10 to be connected to the respective section 201.1, 201.2, 201.3, 201.10 via a feed line section 204.1a, 204.2a, 204.3a, 204.10a by means of a main line connection device 34.1, 34.2, 34.3, 34.10,

for the volume flow setting devices or pressure-reducing devices 45.1, 45.2, 45.3, 45.10 to be connected with the respective volume flow setting device 45, 45.1, 45.2, 45.3, 45.10 arranged behind the respective feed line switching devices 55.1, 55.2, 55.3, 55.10 when viewed from the respective main line connection device 34.1, 34.2, 34.3, 34.10 via a connecting line section 204.1b, 204.2b, 204.3b, 204.10b, and

for providing a test chamber inlet line 204.1c, 204.2c, 204.3c, 204.10c, which fluidically connects a volume flow setting device 45.1, 45.2, 45.3, 45.10 and the test chamber inlet device E of the respective test chamber device 58.1, 58.2, 58.3, 58.10.

By means of a control device functionally connected with each of the feed line switching devices 55.1, 55.2, 55.3, 55.10 and command signals generated by this, the respective feed line switching device 55.1, 55.2, 55.3, 55.10 can be set into a state in which the feed line switching device 55.1, 55.2, 55.3, 55.10 blocks the flow of test gas through the line 201 and it can be set to a position in which the feed line switching device 55.1, 55.2, 55.3, 55.10 is set into a state in which the feed line switching device 55.1, 55.2, 55.3, 55.10 blocks the flow of test gas through the line 204.1, 204.2. Provisions are made according to one embodiment of the present invention for the feed line switching device 55.1, 55.2, 55.3, 55.10 to assume the closed or blocked position in a prestressed or preset state, so that when no command signal is transmitted from the control signal to the feed line switching device 55.1, 55.2, 55.3, 55.10, the feed line switching device is in the closed position and blocks the flow of test gas through the line 204.1, 204.2.

The gas application devices 2 according to the present invention, which are shown in FIGS. 2, 3, 4 and 5, have, on the inlet side, an inlet line 26.1, 26.2, 26.3, 26.4, 26.5, 26.6, 26.7 (reference numbers corresponding to the embodiment shown) connected to a respective test gas connection device 30.1, 30.2, 30.3, 30.4, 30.5, 30.6 (reference numbers corresponding to the embodiment shown) and connection-side switching devices 40.1, 40.2, 40.3, 40.4, 40.5, 40.6, 40.7 (reference numbers corresponding to the embodiment shown) for setting switching states, with which blocking or letting through of test gas flowing from the respective test gas connection device 30.1, 30.2, 30.3, 30.4, 30.5, 30.6, 30.7 can be set, so that a predetermined test chamber device 58.1, 58.2, 58.10 can be selected, in which a gas can be fed from a selectable test gas container of the selected test chamber device 58.1, 58.2, 58.10. The setting of switching states is performed according to the present invention preferably by means of a control device, which is functionally connected with the connection-side switching devices provided depending on the embodiment. By means of a control device functionally connected with the switch 80, the switch can be moved into a closed position, in which the switch 80 of the respective switching device 40.1, 40.2, 40.3, 40.4, 40.5, 40.6, 40.7 is set into a state in which the switching device 40.1, 40.2, 40.3, 40.4, 40.5, 40.6, 40.7 blocks the flow of test gas through the line 201, and into an open position, in which the switch 80 sets the switching device 40.1, 40.2, 40.3, 40.4, 40.5, 40.6, 40.7 into a state in which the switching device 40 blocks the flow of test gas through the line 201. Provisions are made according to one embodiment of the present invention for the switch 80 to assume the closed position in a prestressed or preset state, so that when no command signal is transmitted from the control device to the switch 80, the switch is in the closed position and blocks the flow of test gas through the line 201.

In the embodiments of the gas application devices 2 according to the present invention, which are shown in FIGS. 2, 3, 4 and 5, the connection-side switching devices 40.1, 40.2, 40.3, 40.4, 40.5, 40.6, 40.7 are connected with the connection line 201a via a connection line 28.1, 28.2, 28.3, 28.4, 28.5, 28.6, 28.7 each and via a connection device 29.1, 29.2, 29.3, 29.4, 29.5, 29.6, 29.7 each.

As is shown in the embodiment of the gas application device 2 according to the present invention as shown in FIG. 3, at least one pressure-limiting valve 52.1, 52.2, 52.3, 52.7 each, which are connected with a connection-side switching devices 40.1, 40.2, 40.3, 40.4, 40.5, 40.6, 40.7 each especially via a connecting line 27.1, 27.1, 27.3, 27.7 each, may be provided between a test gas connection device 30.1, 30.2, 30.3, 30.4, 30.5, 30.6, 30.7 each and a connection-side switching device 40.1, 40.2, 40.3, 40.7. The connection-side switching devices 40.1, 40.2, 40.3, 40.7 and the pressure-limiting valve 52.1, 52.2, 52.3, 52.7 may be arranged in a gas application device 2 according to the present invention in the reversed order when viewed from the connection-side switching devices 40.1, 40.2, 40.3, 40.7. A two-stage pressure reduction of the gas flowing from a gas container connected to the gas application device 2 or test gas container 11, 12, 13, 14, 15, 16, 17 can be achieved with a pressure-limiting valve 52.1, 52.2, 52.3, 52.7 if a pressure-limiting valve 52.1, 52.2, 52.3, 52.7 is additionally installed in the connection lines 25.1, 25.2, 25.3, 25.4, 25.5, 25.6, 25.7, as this is provided in the gas application device 2 according to FIG. 3.

As is shown in the embodiment of the gas application device 2 according to FIG. 2, it can generally also be possible that no pressure-limiting valve is installed in the connecting line 26.1, 26.2, 26.3, 26.7 or 28.1, 28.2, 28.3, 28.7 or a pressure-limiting valve is installed in some of these lines only.

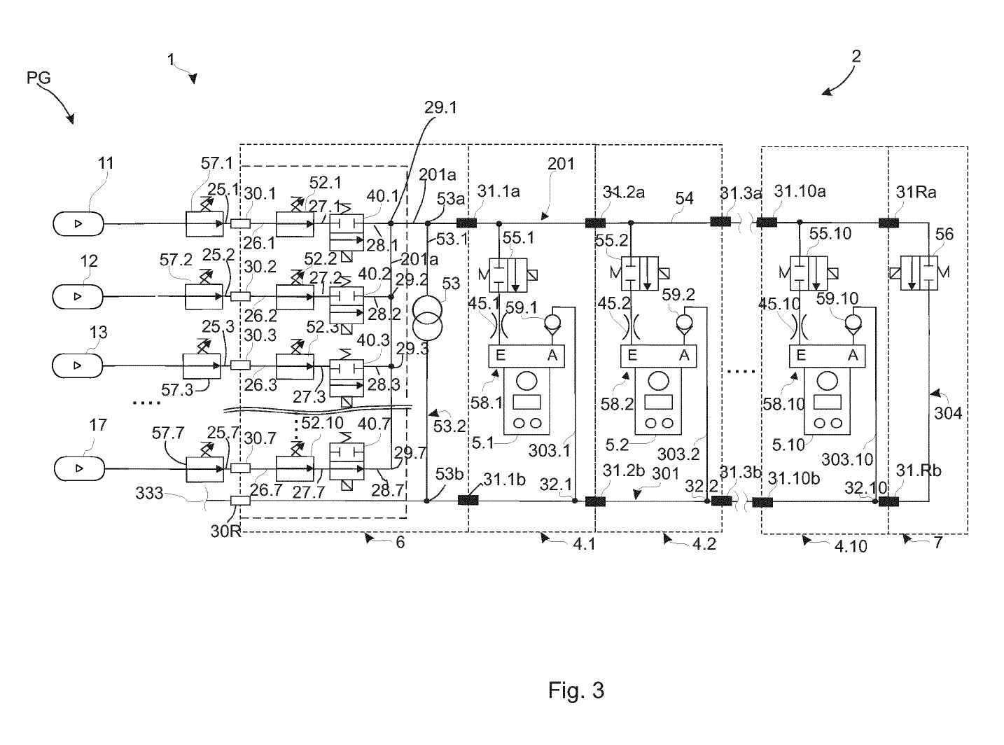

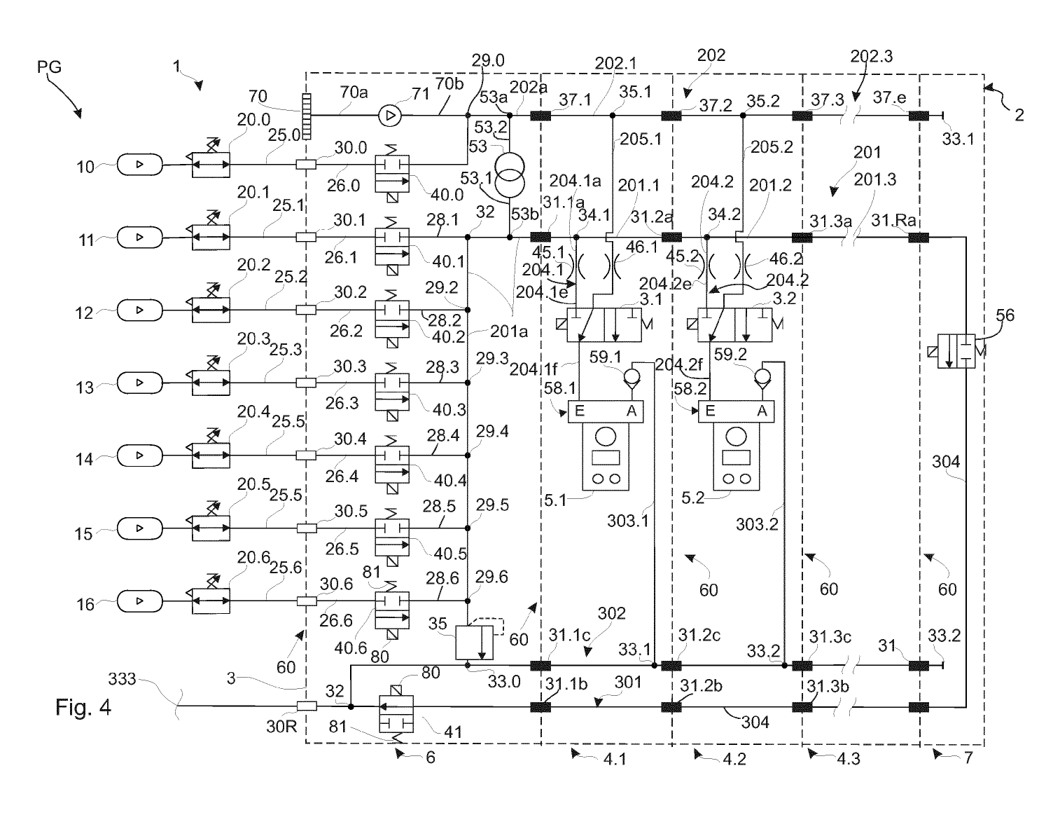

In the embodiments of the gas application device 2 according to the present invention, which are shown in FIGS. 4 and 5, said gas application device has a second main line 202, which are composed of a plurality of line sections 202.1, 202.2, 202.3, which are connected to one another by means of connection devices 37.1, 37.2, 37.3, in case of a modular design of the gas application device 2 with a plurality of test modules 4.1, 4.2, 4.3 connected to one another. The number of line sections of the main line 201 and in the given embodiment of the second main line 202 depends on the number of test modules 4.1, 4.2, 4.3. The end of the row of line sections 202.1, 202.2, 202.3 may be a rear connection device 37.e, via which gas can flow out of the second main line 202 and the gas application device 2, and the receiving container connected with the rear connection device 37.e may be provided for this (not shown in FIGS. 4 and 5). As an alternative or in addition, provisions may be made for the second main line 202 to be fluidically connected with the recirculating line 304 especially via a rear connection device 37.e.

As is shown in FIGS. 4 and 5, provisions may be made when connecting test gas containers to connection devices 30.1, 30.2, 30.3, 30.7 for the test gas containers 10 through 16 to be provided with a settable pressure regulating valve 20.1 through 20.7, which is screwed onto the test gas containers by means of a thread device. The pressure regulating valves 20.1 through 20.7 regulate the pressure present in the test gas containers, which may be between 30 bar and 150 bar and equals, e.g., 100 hPa, to a predetermined pressure, which is especially 0.5 bar and is especially in the range of 0.3 bar to 5 bar. It is guaranteed with these means that the test gas containers 1 through 10 provide a gas flow of the respective test gas for the respective main line with a predetermined gas pressure over a predetermined time during release.

As an alternative, the test gas containers 10 through 16 may be provided with a settable or non-settable pressure-limiting valve.

A further embodiment of a test module is shown with the test modules 4.1, 4.2 in FIGS. 4 and 5. This has:

a feed line switching device 3.1, 3.2, whose first inlet is connected to a first feed line 204.1, 204.2, whose second inlet is connected to a second feed line 205.1, 205.2, and whose outlet is connected to a test chamber inlet line 204.1f, 204.2f, wherein the first feed line 204.1, 204.2 is connected to a corresponding section 201.1, 201.2 of the first main line 201 by means of a main line connection device 34.1, 34.2, and wherein the second feed line 205.1, 205.2 is connected by means of a second main line connection device 35.1, 35.2 to a corresponding section 202.1, 202.2 of the second main line 202, wherein the test chamber inlet line 204.1f, 204.2f is connected to an inlet device E of the test chamber device 58.1, 58.2,

a volume flow setting device or pressure-reducing device 45.1, 45.2, which is installed in the feed line section 204.1, 204.2 and is connected via a connection line 204.1d, 204.2d to a corresponding section 201.1, 201.2 of the first main line 201, wherein the pressure-reducing device 45.1, 45.2 is connected fluidically with a first inlet device of the respective feed line switching device 3.1, 3.2 by means of a respective feed line section 204.1e, 204.1e, and

a volume flow setting device or pressure-reducing device 46.1, 46.2 installed in the respective feed line section 205.1, 205.2.

A control device,

which can generate command signals, on the basis of which the respective feed line switching device 3.1, 3.2 can be set into a state in which the feed line switching device 3.1, 3.2 blocks the flow of test gas through the line 204.1, 204.2 or 205.1, 205.2, and into a position, in which the feed line switching device 3.1, 3.2 permits a flow of test gas through the line 204.1, 204.2 or 205.1, 205.2, wherein provisions may be made, in particular, for the feed line switching device 3.1, 3.2 to assume a closed or blocked position in a prestressed or preset state, so that when no command signal is transmitted from the control device to the feed line switching device 3.1, 3.2, the latter is in the closed position and blocks the flow of test gas into the test chamber inlet line 204.1f, 204.2f, and

which can generate the command signals, on the basis of which the admission of gas to the test chamber inlet line 204.1f, 204.2f is permitted from the feed line section 204.1, 204.2 or from the feed line section 205.1, 205.2 as desired,

is functionally connected by means of a with each of the [sic—Tr.Ed.] feed line switching devices 3.1, 3.2.

In the embodiment of the gas application device 2 according to the present invention according to FIG. 4, this has a connection device 30.3, to which a gas container 10, which may be especially an inert gas container or neutral gas container, can be connected, especially in addition to the test gas containers (reference numbers 11, 12, 13, 14, 15, 16 depending on the figure). A pressure-regulating valve 20.0 or a pressure-limiting device may be connected to the outlet of said gas container. The gas container 10 or optionally the pressure-regulating valve 20.0 or the pressure-limiting device is connected to a connection device 30.0 of the gas application device 2 via a connection line 25.0. A connection-side switching device 40.0 is connected to the test gas connection device 3.0 via an inlet line 26.0, and a connecting line 82.0 with a line connection 29.0 is, in turn, connected to said switching device 40.0, and the line 28.0 is connected to the main line 202 with or at the said line connection 29.0. Thus, especially a test gas cylinder containing fresh air or inert gas can thus be connected via the connection device 30.0 for purging or neutralizing the lines and especially the test modules. Depending on the test gas used, it may be necessary not to feed fresh air from the ambient air for purging or neutralizing the lines, but a corresponding inert gas, which does not react with a gas mixture present in the test gas chamber device 58. The test gas chamber device can thus be purged with the inert gas provided in the second main line 202 and a zero gas calibration of the test specimen can thus be carried out.

As an alternative or in addition to the connection of the line 28.0 to the second main line, an access 70 or an opening of a connection line 70a to a space containing ambient air or to a container containing ambient air may also be formed on the second main line 202. This container may have especially a filter for filtering out components of the ambient air. To feed ambient air to the second main line 202, a pump 71, which can draw in ambient air through the opening 70a and feed same to the second main line 202 via a connecting line 70b, is connected to the connection line 70a.

By providing both the connection device 30.0 for connecting a neutral gas or inert gas container with the connecting line 28.0 and the pump 71 with the opening 70 and the connecting line 70b, ambient air or inert gas can be fed as desired to the test chamber devices of a gas application device 2.

In the embodiment of the gas application device 2 according to the present invention according to FIG. 5, this likewise has a connection device 30.0, to which a gas container 10, which may be especially an inert gas container or neutral gas container, can be connected, especially in addition to the test gas containers (reference numbers 11, 12, 13, 14, 15, 16 depending on the figure). Compared to the embodiment of the gas application device 2, according to the present invention shown in FIG. 4, the lines 70b and 26.0 are connected to an on-off valve, with which ambient air can be fed via the line 70b or inert gas or neutral gas via the line 26.1 as desired corresponding to the particular set switching position of the second main line 202 by manual actuation and/or especially electric actuation thereof, e.g., by means of the control device.

A differential pressure sensor 53, which is placed with differential pressure lines 53.1 and 53.2 between the two main lines 201 and 202 and is connected to same in order to determine the differential pressure, which is obtained from the pressure prevailing in the first main line 201 and the pressure prevailing in the second main line 202, is provided in the embodiments according to FIGS. 4 and 5. The connecting lines of the operating module 6 and the test modules 4.1 through 4.10 connected thereto can be checked for tightness with the differential pressure sensor 53, so that the tightness of the device and of the correct connection of the test modules 4.1 to the operating module 6 can be monitored.

A nonreturn valve 59 can be provided, in general, in the embodiments on an outlet line 303, which is connected with the test gas outlet A in a fluid-tight manner, in order to prevent the undesired escape of the test gas from the test gas outlet A of the test chamber device 58. The gas application device 2 may have a gas recirculating module 7, which has a connecting line 304, which can be connected to the main line 201 and the recirculating line 301 and which fluidically connects the main line 201 with the recirculating line 301.

Modes of operation of embodiments of the gas application device 2 according to the present invention will be described below:

When a test gas container PG is connected to the gas application device 2 together with the pressure-limiting valve 57 in the embodiments of the gas application device 2 according to FIG. 1a, a gas pressure that is reduced by the pressure-limiting valve 57 compared to the pressure in the test gas container is present on the inlet side, i.e., at the connection device 30. When a test gas container PG is connected, together with the pressure-limiting valve 57, to the gas application device 2 in the embodiments of the gas application devices 2 according to FIG. 1b, the pressure prevailing in the test gas container is present on the inlet side, i.e., at the connection device 30. This pressure is reduced by a pressure-limiting valve 52 installed in the main line 201 within the gas application device 2 or a pressure-limiting device 52 relative to the pressure prevailing in the test gas container.

A switching device 40 functionally connected with the control device or input device and/or a switching device 55 functionally connected with the control device or input device may be optionally provided in both embodiments of the gas application device 2. To perform a testing of a gas measuring apparatus 5 and especially of a sensor device, a test gas container PG of a predetermined gas species is connected to the gas application device 2 with or without pressure-limiting valve 57, with which gas species a sensor signal of the sensor device can be expected for measuring a predetermined property, such as a partial gas concentration in a predetermined area. When a testing device 5 is connected to a gas measuring apparatus accommodating device, a test is performed to determine whether the testing device generates actual measured signals, from which it can be determined or indicated that the testing device actually assigns the predetermined property to the predetermined desired value range. In the case of the switching device 40 and/or of the switching device 55, the respective switching device is commanded by means of the control device and/or input device functionally connected with the respective switching device 40, 55 such that this switches to its flow state, in which the test gas flows through the main line 201 and/or the feed line 204. A continuous flow of the test gas with maximum throughput and more or less constant pressure, provided by the pressure-limiting device 52 and/or the pressure-limiting device 57, is guaranteed with the volume flow setting device 45 provided in the test chamber feed line 204. In the embodiment shown in FIG. 1, the volume flow setting device 45 is a throttle valve, which forms a narrowing in the feed line 204 with a predetermined throttling cross section in order to generate a pressure drop. The throttling cross section of the throttle valve 45 may be able to be set or also unchangeable.

A plurality of test gas containers 10 through 16 are connected in the embodiment of the gas application device 2 according to the present invention according to FIGS. 2 through 5, and the number of said gas application devices is determined by the number of gas species or test gas containers needed for a testing device measurement procedure. If a plurality of gas measuring apparatuses 5 shall be tested in one testing operation, a plurality of test modules 4.1 through 4.10 can be connected one after another to the connections 31.2a and 31.2b, 31.3a and 31.3b, etc., so that a plurality of gas measuring apparatuses 5 can be connected to the main line 201 at one time. Connection-side switching devices 40.1, 40.2, 40.3, 40.4, 40.5, 40.6, 40.7 (reference numbers according to the figures) can be brought by means of a control device, which may be embodied together with an input and/or display device in the control module 6, into a state in which test gas flowing from the respective test gas connection device 30.1, 30.2, 30.3, 30.4, 30.5, 30.6, 30.7 is allowed to flow through these switching devices, so that the test gas is provided to the test chamber devices 58 and the respective gas measuring apparatuses 5 connected to these from one or more test gas containers 11, 12, 13, 14, 15. The described testing of the gas measuring apparatuses 5 connected to the test chamber devices 58 will then take place.

Provisions may be made for gas measuring apparatuses 5 to be uncoupled from test chamber devices 58, 58.1, 58.2, 58.3, 58.10 and for other gas measuring apparatuses 5 that are to be tested to be connected to test chamber devices on which the testing operation of the respective gas measuring apparatus 5 has been concluded, while gas measuring apparatuses 5 remain connected to certain test chamber devices 58, 58.1, 58.2, 58.3, 58.10 and no other gas measuring apparatuses 5 are connected to certain test chamber devices on which the testing operation of the respective gas measuring apparatus 5 has not yet been concluded.

The master module or operating module or control module 6, which is shown in FIGS. 2 through 5, may be designed as a valve block 400 comprising a plurality of feed switching devices 40.1-40.7, especially in the design in the form of 2/2-way valves, which are actuated by the control device of the master module 6 and/or by the individual control devices of the test modules 4.1 through 4.10 and are brought into desired states.

The individual feed lines 204.1 are connected by branches or connection elements 32 with the main line 201 of the operating module/master module 6, which ends in a module connection device 31 at an end of the device limit facing away from the connection side 7. The master module 6 has, further, a section of a recirculating line 301, which is provided, on the one hand, with a connection bushing 30 and, on the other hand, with a module connection device 31.1, and the collection main line 201 and the recirculating line 301 of test modules 4.1 through 4.10 that can be coupled can be connected by means of the module connection devices 31.1 and 31.2.

A plurality of test modules 4.1-4.10, which can be coupled with one another, for which corresponding module connection devices 31 are provided in the respective module on the suitable side of the test module, are shown in the gas application system 1 shown in FIG. 2 and in the gas application device 2 shown in FIG. 2.

It is seen in embodiments of the gas application device 2 according to the present invention according to FIGS. 2 through 5 that a nonreturn valve 59.1 through 59.10, which can prevent test gas from flowing off at the test gas outlet A of the test chamber device 58, is inserted at the outlet or removal line 303.1 through 303.10 in embodiments of the gas application device 2 according to the present invention according to FIGS. 2 through 5. Further, at least one pressure-reducing device in the form of a throttle valve 45.1 through 45.10, which ensures a continuous flow of test gas into the testing chamber and can reduce the pressure built up in the main line 301 by the test gas, e.g., from 0.5 hPa to 1 Pa, is provided at the inlet E of the test chamber device 58, which is in connection with the feed line 103.

To make possible the outflow of unneeded test gas from the main line 201 via another way rather than through the test chamber device 58 and to make it possible to purge the main line, a gas recirculation module 7 is provided, which likewise has a recirculation switching device 56 especially in the form of a switching device 56. If the switching devices 55.1 through 55.10 of the test modules are closed, a purging gas can be fed into the main line 201 in order to clean the main line 201. Provisions are made for this for feeding a purging gas, which is fresh air or inert gas, from one of the test containers 10 into the main line 201, and the switching device 40.1 and the recirculation switching device 56 or the 2/2-way valve 56 are in an open state, and all other 2/2-way valves of the master module 6 and of the test modules 4.1 through 4.10 are in a closed state, so that the test gas still present in the main line 201 can be purged by the purging gas and is flushed out of the gas application device 2 via the recirculating main line 301, for which purpose a suitable receiving container, which is not shown in FIG. 2, may be provided for this at the outlet point 333.

The gas recirculation module 7 may also be eliminated if the module connection devices 21 at the last testing apparatus module 4.10 are provided with suitable closing plugs, so that the test gas and purging gas can flow only from the main line 201 via the feed line 204.1 into the test chamber device 58.1 through 58.10 and the outlet line 303.1 through 303.10 to the recirculation line 301 when the respective feed switching device 55.1 through 55.10 is switched into an opened or flow state.

FIG. 3 shows another pneumatic circuit diagram of another embodiment of the gas application device 2 according to the present invention. The embodiment of the device 1 according to the present invention for testing gas measuring apparatuses or test specimens 5, 5.1-5.10 shown in FIG. 3 is also designed for a modular design. The module device limits of the respective modules 6, 4.1, 4.2-4.10 and 7 are indicated by the broken lines 60. Just as in the embodiment according to FIG. 2, the embodiment of the gas application device 2 for testing gas measuring apparatuses or for calibrating same, which is shown in FIG. 3, also has connection devices 30.1 through 30.7, to which a plurality of test gas containers or gas storage devices 10 through 16 can be connected via a gas connection line 25.0 through 25.7, on the connection side 7 of the master or operating module 6. Further, the gas storage device 10 through 16, which is provided with a pressure-limiting valve 57.1 through 57.7, is arranged in the connection side 7. The pressure-limiting valve 57.1 through 57.7 may be either self-controlling, as in FIG. 1, or it is actuated via the operating module 6 or the testing apparatus module 4.1. It is possible to provide either three connection devices 30 on the connection side 7 of the operating module 6, or, as is shown in FIG. 2, six connection devices 30.1 through 30.7, in which case at least one test gas cylinder is a fresh air test gas cylinder or an inert gas test gas cylinder, which contains an inert gas or fresh air gas for purging the main lines and the test chambers of the respective testing apparatus modules.

The exemplary embodiment of the gas application device 2 shown in FIG. 3 differs from the gas application device 2 shown in FIG. 2 in that additional connection-side switching devices 40.1, 40.2, 40.3, 40.4, 40.5, 40.6, 40.7 are provided in the master module 6 in the feed lines 26.1 through 26.7 in order to make it possible to additionally reduce the gas pressure provided in the test gas containers 1 through 10 to a predetermined pressure independently from the pressure-limiting valves 57.1 through 57.7.

The test modules 4.1 and 4.2 shown in FIG. 3 have essentially the same design as the test modules 4.1, 4.2, and the test chamber device 58 is designed in both test modules 4.1 and 4.2 such that different gas warning devices or test specimens 5 can be inserted for calibration and testing. Further, a third test module 4.3 can be connected to the test module 4.2 via the module connection devices 31, and a fourth test module 4.4 can, in turn, be connected to said third test module 4.3 in the same manner. Up to 10 test modules can be coupled with one another in this manner, and a larger number of test modules may also be coupled with one another. The individual test modules 4.1 through 4.10 are always designed such that one test module can be coupled with the other test module. Module connection devices 31, by means of which the main lines 201 and 202 can be coupled with one another, are provided for this, and the number of test modules that can be coupled via the module connection devices 31 is not greater than 10 in the example being shown, but the case in which the number of the plurality of modules may be greater than 10 is also within the scope of the present invention, and there is no theoretical limitation to the possible number of test modules 4 that can be coupled. The only limitation of the possible number of test modules 4.1-4.n that can be coupled is dictated by practical feasibility.

One peculiarity of the embodiment shown in FIG. 4 is that there is a first main line 201, with which test gas is preferably provided for the individual test modules 4.1 through 4.10, and that there is a second main line 202, with which purging gas or inert gas or ambient air is preferably provided. The test gas provided in the first main line 201 and the inert gas provided in the second main line or the ambient air provided may be polled, as needed, from the respective test modules 4.1 through 4.10. Further, a differential pressure sensor 53 is arranged between the two main lines 201 and 202 for testing the connecting lines of the operating module 6 and the test modules 4.1 through 4.10 for tightness, so that the tightness of the device and of the correct connection of the test modules 4.1 to the operating module 6 can be monitored.

Further, the test gas containers 10 through 16 according to FIG. 4, which contain a certain test gas, for example, H₂S, CO₂ or CO, are provided with a settable pressure regulating valve 20.1 through 20.7, which has been screwed onto the test gas containers 10 through 16 by means of a thread device. The pressure regulating valves 20.1 through 20.7 regulate the pressure present in the test gas containers, usually 100 hPa, down to a predetermined pressure, in this case 0.5 hPa, and ensure that the gas flow from the test containers 1 through 10 is maintained at the predetermined gas pressure over a predetermined time during the release of the test gas from same.

It is seen in FIG. 4 that the respective main lines 201, 202 are formed by the individual main line sections 201.1 through 201.10, 202.1 through 202.10 of the test modules 4.1 through 4.10, which said sections are coupled with one another, and by the main line sections 201a and 202a of the test module 6. As was mentioned already, module connection devices 31 are provided in the respective test module 4.1 and 4.2 and the operating or master module 6, so that a fluid-tight main lines 201, 202 can always be formed.

Further, it can be seen from FIG. 4 that a 2/2-way valve 41, which can be actuated electromagnetically, as it is indicated by symbol 80, and can be reset by means of an adjusting spring 81 into the starting position shown, is inserted in the feed lines 26.0 through 26.10 of the operating module 6 for a group of connection-side switching devices 40.1 through 40.7 and in the recirculating main line 302. A group of connection devices 30, via which different test gas cylinders 10 through 16 can be connected by means of a connection line 25.0 through 25.7, can be connected to the group of directional control valves 40.1 through 40.7 on the test gas container connection side 7, and a settable pressure regulating valve 20.1 through 20.7 can be inserted in each connection line 25.0 through 25.7. Further, a differential pressure sensor 53, which can determine the differential pressure present between the respective main lines 201 and 202 by a differential pressure measurement, is connected to the first main line 201 and the second main line 202. In addition, an overpressure-limiting valve 35, which is switched to flow at a predetermined overpressure in the main line 201 in order to protect the main line 201 from an excessively high gas pressure, is inserted between the main line 201 and the recirculating main line 302. The overpressure-limiting valve 35 may be a self-actuating overpressure-limiting valve 35, in which case direct actuation is performed via the internal control of the pressure-limiting valve 35, or the overpressure-limiting valve 35 may be an overpressure-limiting valve 35, in which case the pressure-limiting valve 35 is controlled or actuated outside the pressure-limiting valve 35, especially in the master or operating module 6 and/or the coupled test modules 4.1 through 4.10.

According to a preferred embodiment, all modules 4.1 through 4.10 are supplied with the respective test gas and inert gas simultaneously via the two separate main lines 201 and 202. Test gases are sent to the respective modules 4.1 through 4.10 via the main line 201 from the respective test gas cylinders, and the inert gas or zero gas is provided via the main line 202. By means of the 3/2-way valve 3 in the respective modules 4.1 and 4.2, the individual module 4.1 through 4.0 can fall back on the requested gas, either test gas or inert gas, individually and feed it to the respective test specimen 5.1 through 5.10 or 5.1 through 5.n via the test chamber device 58, where n is a natural number greater than 10.

Six test gas inlets 30.1 through 30.6, to which corresponding test gas cylinders 11-16 can be connected, are embodied n the pneumatic circuit diagram of an exemplary embodiment of the gas application device 2 shown in FIG. 4. A test gas cylinder containing fresh air or inert gas can be connected via a connection bushing. Depending on the test gas used, it may be necessary not to feed fresh air from the ambient air but a corresponding inert gas, which does not react with the gas mixture present in the test chamber device 58 to purge the test gas chamber device and thus to make a zero gas calibration of the test specimen possible.

The test gas cylinder 10 may consequently contain a corresponding inert gas or fresh air as described. Fresh air may also be fed to the inert gas main line 202; this happens via a filter 70, which is connected with a pump 71, which draws in ambient air of the operating module 6 and can feed it to the respective feed line via the inert gas main line 202.

The exemplary embodiment of the gas application device 2 shown in FIG. 5 differs essentially in the circuitry of the feed line 26.0 to feed a purging gas from the test gas container 10 with the feed line 70a, at which a pump 71 is provided for drawing in ambient air. The switching between two feed possibilities is provided via the control of the master module 6 and/or the control of the test modules 4.1 through 4.10 coupled with the master module 6. A 3/2-way valve is designed as a feed line switching device via which the second main line 202 can be supplied as desired with ambient air via the feed line 70a or with the contents of the gas container 10 via the corresponding switching state thereof. The gas container 10 may contain a corresponding, suitable gas for purging the test chamber devices 58, for example, inert gas, which may contain especially nitrogen N or one of the noble gases.

The embodiments of the gas application devices 2 shown in FIGS. 4 and 5 have two main lines 201, 202 and corresponding recirculating lines 302, 301, wherein one main line 201 is intended to provide and feed gas for purging and the other main line 202 for providing and feeding test gas. The main line 202 for purging is coupled with a fresh air gas container 10, and the purging gas may also be fed from the ambient air via a pump 7, in front of which a dirt filter may be arranged, if the ambient air is to be used to purge the test chamber device 58 to which a test gas was previously admitted.

According to the present invention, the test modules 4.1, 4.2, 4.3, etc., may fall back each separately and alternatingly on the main line 201 and the main line 202. A feed switching device 3.1 or 3.3, especially in the form of a 3/2-way valve, is provided for this, which is controlled electrically and can be moved into the blocked position by means of a resetting spring. The 3/2-way valve is shown in FIGS. 4 and 5 in a position in which the admission via the feed lines 204.1, 204.2, etc., from the main line 201 towards the test chamber device 58 is blocked, and the admission of gas from the main line 202 into the test chamber device 58 is possible. The respective test chamber device 58.1 through 58.2 is connected for this with the main line 202 via the second feed line 205.1, 205.2, etc., and the third feed line 206.1, 206.2, etc., and the 3/2-way valve 3.1 is shown in a flow position in which gas can flow from the main line 202 into the test chamber device 58.

In the embodiment of the present invention with a test module 4.1, a second test module 4.2 or a gas-recirculating module 7 can be connected to this [module] by means of the module connection devices. When a second test module 4.2 is connected, provisions may be made according to the present invention for the second test module 4.2 to report with a communication device at the operating module or master module and especially the control device thereof when the module devices are coupled with one another such that a flow of gas is possible between the main line and the recirculating line with the gas-recirculating module 7. The test modules 4.1, 4.2, etc., may also be designed each such that these can be actuated independently from the master module 6 and/or the test modules 4.1, 4.2, etc., i.e., e.g., by manual action. As an alternative, provisions may be made for the test modules 4.1, 4.2, etc., to be able to be commanded either by the control unit of the respective test module or by the control unit of the master module 6 via corresponding communication devices.

It is also possible with the gas application devices 2 shown in FIGS. 1a, 1b and 2 through 5 to measure the admission pressure provided by every individual test gas container PG, 11 through 17 or 10 through 16 and the corresponding pressure-regulating valve 20.1 through 20.7 (FIGS. 1b, 4, 5) or by the pressure-reducing device 57 or 57.1 through 57.7 (FIGS. 1a, 2, 3). It can thus be determined whether the admission pressure of the respective test gas container PG, 10, 11 through 16 has been reduced to a predetermined gas pressure of, e.g., 0.5 hPa. The feed switching devices or 2/2-way valves 40, 40.1 through 40.7 are to be brought for this into the flow position, so that test gas can flow into one of the main lines 201, 202 and the pressure in the respective main line 201 or 202 can be measured by means of the respective differential pressure meter 53 provided. For example, one of the feed switching devices, e.g., the feed switching device 40.0 (FIG. 4), can be switched into a flow position, so that test gas can flow from the test gas container 10 into the feed line 26.0 and into the main line 201 or 202, so that the differential pressure sensor 53 connected thereto can measure the differential pressure present between the main lines 201 and 202. The admission pressure, which is always generated by a test gas container PG, 10, 11 through 16 in the feed lines 26, 26.0, 26.1 through 26.2, can be measured in this manner.

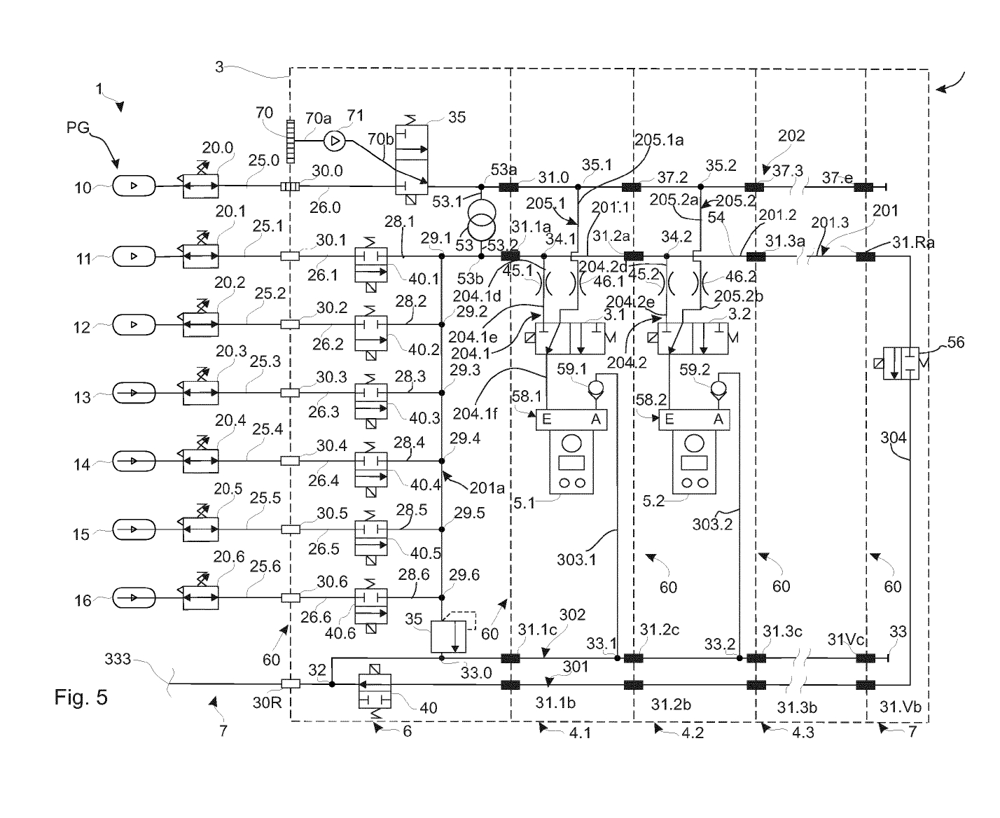

The pressure profile curve 90, which indicates, as is shown in FIG. 6, the admission pressure, for example, of the test gas containers PG, 11, 12, 13, 14, 15, 16, 17 (reference numbers according to the figures), which was recorded with a differential pressure sensor 53 in the gas application device 2, is obtained in a pressure-time coordinate system by the test container secondary measurement. The test gas cylinders can thus be tested for proper connection before the individual gas testing and measuring apparatuses 5.1, 5.2, 5.10 are tested.

A tightness measurement, which can be carried out with the gas application device 2 according to the present invention as a self-test function, will be described below:

One embodiment of the first tightness measurement is described on the basis of the embodiment of the gas application device 2 according to the present invention shown in the figures: An analyzing device is connected functionally with the differential pressure sensor 53 for this. The curve of the differential pressure over time or the changes in the differential pressure after the end of predetermined time periods can be detected and determined with this analyzing device by means of a timer thereof. The change in the differential pressure or the comparison of the actually occurring differential pressure with a desired differential pressure curve can be displayed visually and/or an analysis function determines the deviation of the actual differential pressure from the desired differential pressure by comparison. A leakage value is generated for the gas application device 2 with such a determination of a change in the differential pressure over a time period beyond a predetermined maximum to assign a leakage state to the gas application device 2. The limit for the assignment of the leakage to a gas application device 2 may be, e.g., a pressure drop by a percentage of a first actual value and, e.g., a value between 1% and 15% after one minute. Provisions may also be made for a plurality of measurement time periods, i.e., two or three time periods, to be checked one after another with respect to a minimum pressure drop.

If leakage is detected, provisions may be made for the test function to generate a leakage status signal, with which it is indicated that the gas application device 2 is leaky. Otherwise, a signal can be generated, which corresponds to the leakage state.

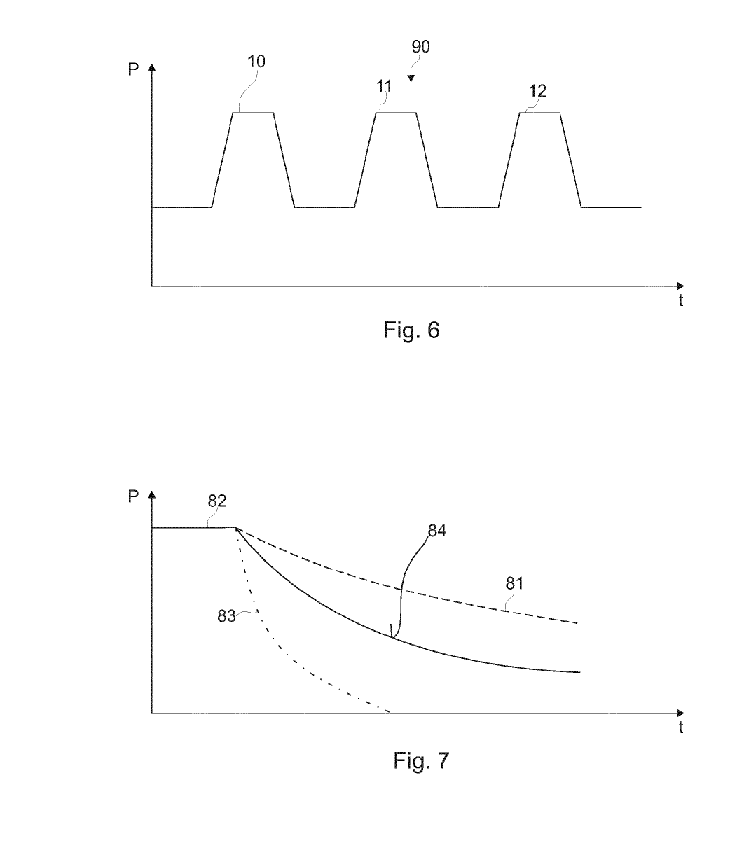

To carry out the tightness measurement, the gas containers PG, 10, 11 through 16 are connected to the gas application device 2 and the main line 201 as well as optionally the other main line 202 is/are filled at first with a test gas from the respective test gas container 11 through 16. The connection-side switching devices 40, 40.1, 40.2, 40.7 and the recirculation switching device 56 and 55 as well as optionally a switching device (not shown) at the end of the other main line 202 (behind the connection point 37.e) and optionally the respective at least one feed line switching device 55, 55.1, 55.2, 55.10 provided are brought into a blocked position, so that the main line 201 and optionally the other main line 202 are blocked downstream of the feed switching devices 40 and the gas present in the main line 201 and optionally in the other main line 202 cannot flow through the test chamber device 58, 58.1, 58.2, 58.3, 58.10, nor can it flow off into the recirculating line 304. The pressure in the main line 201 can be determined with a pressure sensor, i.e., especially with the differential pressure sensor shown in the respective figures. The curve section 82 shown in FIG. 7 is obtained as the pressure curve in this switching state insofar as the gas application device 2 is intact. The pressure is plotted, for example, in hPa on the vertical axis and the time t is plotted, for example, in seconds on the horizontal axis in FIG. 7. The feed line switching device 55, 55.1, 55.2, 55.10 is subsequently opened. Depending on the particular given state of the gas application device 2, e.g., intactness or defective state of the gas application device 2, the measured curves 81, 83 or 84 according to FIG. 5 are obtained from the pressure measurement, e.g., with the differential pressure sensor 53.

If the gas application device 2 is intact, curve 82 can indicate a pressure drop and thus a declining curve in FIG. 7 if the main line 201 or the pneumatic components pneumatically connected with the main line 201, which are [located] upstream of the feed line switching device 55, 55.1, 55.2, 55.10 (FIGS. 1a, 1b, 2, 3) or 3.1, 3.2 (FIGS. 5, 6) in relation to the gas flow in the gas application device 2, are leaky.

FIG. 7 shows the curve describing the pressure drop for different cases, and depending on whether a defect is present in the gas application device 2 according to the present invention or not:

The curve designated by reference number 84 shows a curve of a differential pressure detected by the respective differential pressure sensor, which represents a desired curve relative to the respective gas application device 2 in question;

The curve designated by reference number 81 shows a curve of a differential pressure detected by the respective differential pressure sensor, which curve deviates from the desired curve according to curve 84, which occurs in the case in which the pressure-reducing device 45, 45.1, 45.2, 45.3, . . . , 45.10 is partially closed in an unallowable manner, which may be caused especially by contamination of the pressure-reducing device 45, 45.1, 45.2, 45.3, . . . , 45.10 or by kinking of the feed line 204.1c; 204.1f, 204.2f or by contamination;

The curve designated by reference number 83 shows a curve of a differential pressure detected by the respective differential pressure sensor, which deviates from the desired curve according to curve 84 in an unacceptable manner, which occurs in a case in which a connecting line, especially line 204.1, 204.2 or 204.10 is interrupted and is separated from this or broken off at one of its connection points.

An unacceptable deviation from a curve 84 can be detected and optionally also displayed in one embodiment of the gas application device 2 according to the present invention by means of an analysis function, which is linked functionally especially with the respective pressure sensor or differential pressure sensor 53.

The gas application device 2 according to the present invention can be operated according to the following method:

Providing a test gas at a test gas connection device 30 for connecting a test gas container PG, 10 through 16 (reference numbers depending on the figures) containing a test gas;

Optionally reduction of the test gas pressure to a predetermined pressure by means of a pressure-reducing device 20, 20.1 through 20.10;

Opening at least one feed line 26, 26.1 through 26.10, which connects with a main line 201, so that test gas is provided in the main line with a predetermined pressure;

Opening at least one first feed line 45, 45.1 through 45.10, which is connected to a main line 201 and to a test chamber device 58, so that the test gas provided in the main line 201 can flow into the test chamber of the test chamber device 58;

Throttling the at least one first feed line 45, 45.1 through 45.10, which is connected to a main line 201 and to a test chamber device 58, so that the test gas provided in the main line 201 can flow into the test chamber of the test chamber device 58, wherein throttling is defined as the setting to a predetermined volume flow at a constant admission pressure;

Admission of a predetermined gas to the test chamber of the test chamber device 58 over a predetermined time period and/or until a predetermined test chamber pressure builds up in the test chamber of the test chamber device; and

Removal of the test gas from the test chamber of the test chamber device 58 when the predetermined gas admission time has come to an end and/or the predetermined test chamber pressure has been reached in the test chamber of the test chamber device.

While specific embodiments of the invention have been shown and described in detail to illustrate the application of the principles of the invention, it will be understood that the invention may be embodied otherwise without departing from such principles.

1. A gas application device comprising:
a test gas connection device for connecting a test gas container containing a test gas;
a test chamber device with a test chamber and with a gas measuring apparatus accommodating a device for detachably coupling a gas measuring apparatus for detecting a property of a test gas flowing through the test chamber device by means of a test gas sensor device of the gas measuring apparatus, and feed connection device;
a main line connected with the test gas connection device;
a feed line fluidically connecting the main line and the feed connection device of the test chamber device; and
a volume flow setting device installed in the feed line for setting the volume flow of a test gas provided from the main line to the test chamber, so that when a test gas flowing with a predetermined pressure that is constant within preset limits is provided, this test gas flows in the test chamber device with a predetermined test chamber volume flow that is constant within preset limits.

2. A gas application device in accordance with claim 1, further comprising a pressure-reducing device installed in the main line, so that the test gas is available between the pressure-reducing device and the volume flow setting device with a predetermined pressure that is constant within preset limits.

3. A gas application device comprising:
at least two test gas connection devices each for connecting one test gas container containing a test gas, each of the gas connection devices having a connected pressure-reducing device, wherein the gas application device is for testing gas measuring apparatuses;
at least one test chamber device with a test chamber and with a gas measuring apparatus accommodating device for the detachable connection of a gas measuring apparatus for detecting a property of a test gas flowing through the test chamber device by means of a test gas sensor device of the gas measuring apparatus,
at least two inlet lines, each of the two inlet lines being connected to one of the test gas connection devices;
connection-side switching devices for setting switching states with which blocking or letting through of test gas flowing from the respective test gas connection device can be set, wherein each one of the connection-side switching devices is fluidically installed in a respective one of the inlet lines;
a main line, which is connected to each of the inlet lines, so that the feed of test gas from one or more of the inlet lines into the main line can be set by means of the connection-side switching devices;
at least one feed line, which connects the main line with a feed connection device of the at least one test chamber device; and
at least one volume flow setting device installed fluidically in the at least one feed line for setting the volume flow of a test gas provided from the main line for the respective test chamber to a predetermined test chamber volume flow in the respective test chamber device, so that the at least one test chamber device can be selected, into which a test gas can be fed from a selectable test gas container of the selected test chamber device.

4. A gas application device in accordance with claim 3, further comprising another test chamber device wherein:
an outlet line is connected to each test chamber device, and a nonreturn valve, which permits the discharge of test gas from the respective test chamber only when a predetermined minimum test gas pressure has developed in the test chamber, is installed in each said outlet line.

5. A gas application device in accordance with claim 3, further comprising a commandable feed line connection device for blocking or letting through the flow of test gas flowing from the main line to the at least one test chamber device the feed line connection device being installed fluidically in the at least one feed line, so that a predetermined test chamber device, into which a test gas can be fed from a selectable test gas container of the selected test chamber device, can be selected with a combination of a feed line switching device and a volume flow setting device, which said combination is fluidically installed in one of the feed lines each.

6. A gas application device in accordance with claim 3, further comprising:
a second main line, which is connected to one of the test gas connection devices for introducing an inert gas and/or in combination with a compressor device connected to a surrounding area containing ambient air for admitting ambient air into the second main line.

7. A gas application device in accordance with claim 6, further comprising:
a feed line switching device;
a second feed line connected to the second main line and fluidically in connection with the test chamber of the test chamber device, wherein the first feed line, connected to the first main line, is connected to the feed line switching device and the second feed line is connected to a second inlet of the feed line switching device, with an outlet of the feed line switching device connected to the test chamber device via a test chamber feed line and with the feed line switching device blocking or letting through test gas provided from the first main line or from the second main line, as desired, through the feed line switching device taking place as desired;
a first volume flow setting device for setting a test gas volume flow, provided from the first main line to the test chamber of the test chamber device, chamber in the first feed lines when test gas with a pressure that is constant within predetermined limits is provided in the first main line, wherein the first volume flow setting device is designed for setting a test gas provided from the first main line of the respective test chamber to a predetermined test chamber volume flow into the test chamber device; and
a volume flow setting device for setting a test gas volume flow, provided from the second main line to the test chamber of the test chamber device in the second feed line when test gas with a pressure that is constant within predetermined limits is provided in the second main line, wherein the second volume flow setting device is designed for setting a test gas provided from the second main line of the respective test chamber to a predetermined test chamber volume flow into the respective test chamber device, so that test gas provided from the first or second main line, as desired, can be provided in the respective test chamber device corresponding to the switching state of the feed line switching device to the test chamber of the test chamber device with a test chamber volume flow, that has a value that depends on the pressure with which the test gas, provided from the first main line or from the second main line for the feed line switching device.

8. A gas application device in accordance with claim 6, further comprising:
a first feed line switching device;
a second feed line switching device
a second feed line, connected to the second main line and which are fluidically in connection with the test chamber of the test chamber device, wherein the first feed line is connected to the first main line and is connected to an inlet of the first feed line switching device and the second feed line is connected to an inlet of the second feed line switching device, with outlets connected via a test chamber feed line to a respective inlet of the test chamber of the test chamber device, so that blocking or letting through of test gas provided from the first main line or from the second main line, as desired, through the first feed line switching device and the second feed line switching device can take place, as desired;
a first volume flow setting device for setting a volume flow of a test gas provided from the first main line to the test chamber device in the first feed line when test gas with a pressure that is constant within predetermined limits is provided in the first main line, wherein the first volume flow setting device is designed for setting a test gas provided from the main line for the test chamber to a predetermined test chamber volume flow into the respective test chamber device; and
a second volume flow setting device for setting a volume flow of a test gas provided from the second main line to the test chamber device in the second feed line when test gas with a pressure that is constant within predetermined limits is provided in the second main line, wherein the second volume flow setting device is designed for setting a test gas provided from the main line for the respective test chamber to a predetermined test chamber volume flow in the test chamber device, so that test gas provided from the first or second main line, as desired, is provided in the test chamber device, corresponding to the switching state of the feed line switching device, for the test chamber of the test chamber device with a test chamber volume flow whose value depends on the pressure with which the test gas is provided for the feed line switching device from the first main line or from the second main line.

9. A gas application device in accordance with claim 3, further comprising pressure-reducing devices, at least one of which is installed fluidically in an inlet line connected to a test gas connection device.

10. A gas application device in accordance with claim 9, wherein at least one of the connection-side switching devices, and the pressure-reducing devices installed in the respective inlet line are installed in a housing part or frame part of a master module, so that the switching devices are integrated in a master module that is uniform in terms of hardware.

11. A gas application device in accordance with claim 7, in combination with a gas application system control device to provide a gas application system, wherein the gas application system control device is functionally connected with at least a plurality of the connection-side switching devices, so that the gas container from which test gas can be fed to the respective main line can be selected, and/or which is functionally connected with at least a plurality of the feed line switching devices or the respective feed line switching devices connected to both of the main lines, so that test gas can be fed as desired to one or more of the test chamber devices from at least one gas container.

12. A method for testing gas measuring apparatuses, the method comprising the steps of:
providing a test gas at a test gas connection device by connecting a test gas container, containing a test gas, to the test gas connection device;
reducing the test gas pressure to a predetermined pressure by means of a pressure-reducing device in a main line, wherein the predetermined pressure is maintained with a maximum range of variation of 30%;
switching a feed line switching device, which is installed in at least one first feed line and is connected to the main line and to a test chamber device, to a flow state, so that the test gas provided in the main line can flow through the test chamber of the test chamber device; and
setting the volume flow of the test gas flowing in the at least one feed line, so that the test gas provided in the respective main line flows through the test chamber of the test chamber device with a predetermined volume flow.

13. A method in accordance with claim 12, wherein:
the gas measuring apparatus is functionally in connection with a control device and the gas measuring apparatus sends signal values and/or data for respective detected states of the test gas and especially for respective detected gas concentrations of a gas species contained in the test gas to an analyzing device;
the control device at least one of generates a status signal relating to the predetermined state of the test gas having not been reached when the control device receives signal values and/or data for respective detected states of the test gas, which signal values and/or data indicate that the predetermined state of the test gas has not been reached within a predetermined time period, and switches a feed line switching device, which is installed in at least one first feed line and which is arranged between the main line and the respective test chamber device to which the measuring apparatus to be tested is connected, into a blocked state, in which the test gas provided in the main line cannot flow through the test chamber of the test chamber device.

14. A method in accordance with claim 12, wherein at least one feed line, which is connected to a main line, is opened, so that test gas is provided in the main line with a predetermined pressure.

15. A gas application device according to claim 1, wherein the device forms a calibrating gas measuring apparatus for testing and calibrating gas measuring apparatuses.
